(19) United States
(12) Patent Application Publication
(10) Pub. No.: US 2016/0339133 A1
(43) Pub. Date: Nov. 24, 2016

(54) BATTERY POWERED UV SANITATION SYSTEM

(71) Applicant: George J. Lichtblau, New Canaan, CT (US)

(72) Inventor: George J. Lichtblau, New Canaan, CT (US)

(21) Appl. No.: 14/714,822

(22) Filed: May 18, 2015

(51) Int. Cl. A61L 2/24; A61L 2/10

(52) U.S. Cl. CPC A61L 2/24; A61L 2/10

(57) ABSTRACT

An ultraviolet radiation sanitation system which is portable and which operates on battery power to generate UVC radiation to decontaminate bathrooms on aircraft and other small rooms or enclosed areas. The system includes one or more low pressure high output mercury or amalgam UVC lamps mounted on a housing which contains a battery powered power supply, lamp ballasts and control circuitry. The housing also includes a control panel having controls and indicators for system operation. A motion detector is provided on the housing to shut off the system in the presence of detected motion which would occur by the presence of a person. The housing is mounted on a tripod or other stand to support the system on an aircraft bathroom floor or other location in which the system is to be deployed.

### STATEMENT REGARDING FEDERALLY SPONSORED RESEARCH OR DEVELOPMENT

N/A

### BACKGROUND OF THE INVENTION

It is known that pathogens can be present on surfaces which are not regularly cleaned. This is especially true in bathrooms used by many people as in bathrooms on aircraft. It is also known that UVC radiation is effective in killing or deactivating pathogens in air, water and exposed surfaces. A system for providing UVC radiation to kill pathogens in the air and on radiated surfaces in a room is shown in U.S. Pat. No. 8,791,441 of the same inventor as the present invention.

It would be useful to have an effective and convenient system for effective decontamination of pathogens in aircraft bathrooms and other small rooms or spaces.

### BRIEF SUMMARY OF THE INVENTION

The present invention provides an ultraviolet radiation sanitation system which is portable and which operates on battery power to generate UVC radiation to decontaminate bathrooms on aircraft and other small rooms or enclosed areas. The system includes one or more low pressure high output mercury or amalgam UVC lamps mounted on a housing which contains a battery powered power supply, lamp ballasts and control circuitry. The housing also includes a control panel having controls and indicators for system operation. One or more motion detectors are provided on the housing to shut off the system in the presence of detected motion which would occur by the presence of a person. The UVC lamps are of U shape having connectors on one end that can be plugged into electrical sockets on the housing and which are easily plugged in and out for replacement. Each lamp is covered by a protective sleeve to avoid shattering of the lamp glass in the event of breakage. FEP (Teflon) is preferred because it is UVC transmissive with little attenuation and can easily withstand the operating temperature of the lamps. UVC radiation is provided at a prime wavelength of 253.7 nm (referred to as 254 nm) and sufficient radiation can be provided by the system to decontaminate pathogens including Clostridium difficile in a typical aircraft bathroom in about 3 minutes. The on time of the lamps is so short that self-heating of the UVC lamps has no effect on performance of the system. The housing is mounted on a tripod or other stand to support the system on an aircraft bathroom floor or other location in which the system is to be deployed. The system may be operated by a remote control.

### BRIEF DESCRIPTION OF THE SEVERAL VIEWS OF THE DRAWINGS

The invention will be more fully understood from the following detailed description in conjunction with the drawings in which:

FIG. 1a is a pictorial view of one embodiment of a system in accordance with the invention;

FIG. 1b is an elevation view of the embodiment of FIG. 1a;

FIG. 2 is a block diagram of a system in accordance with the invention; and

FIG. 3 is a schematic of circuitry of the system in accordance with the invention.

### DETAILED DESCRIPTION OF THE INVENTION

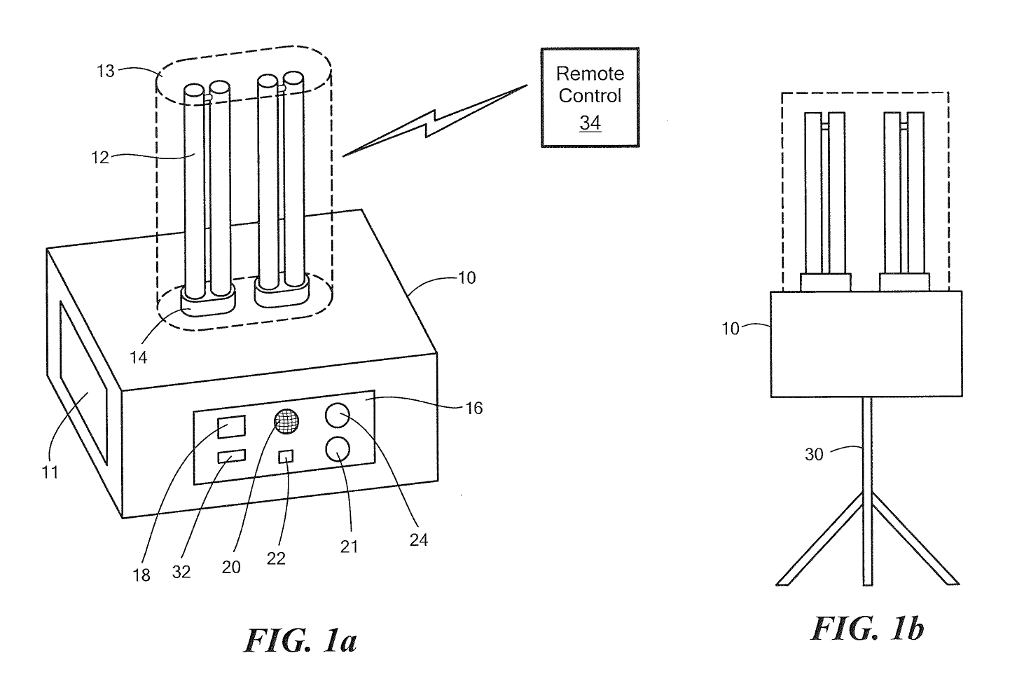

A UVC sanitation system in accordance with the invention is shown in one embodiment in FIGS. 1a and 1b. A housing 10 supports one or more UVC lamps 12 in vertical orientation. Two lamps are illustrated in the embodiment of FIG. 1. The lamps are in the form of U shaped tubes which are pluggable at one end into associated sockets 14 mounted to the top wall of housing 10. The lamps are typically low pressure high output mercury or amalgam UVC generating lamps such as Light Sources, Model LTC55W/2G11/FEP. The lamps are typically about 20 inches in length. Preferably each lamp is covered by a protective sleeve to avoid shattering of the lamp quartz glass in the event of breakage. FEP (Teflon) is preferred because it is UV transmissive with little attenuation and can easily withstand the operating temperature of the lamps. The housing 10 can be made of any suitable material, typically aluminum.

The housing contains one or more lamp ballasts, control circuitry and one or more batteries for powering the system. The ballasts are electronic ballasts and each lamp may be driven by one ballast or a single ballast may drive multiple lamps depending upon the particular lamps and ballasts employed. Battery power is typically 28 volts DC provided by the one or more batteries. The battery or batteries are typically rechargeable and may be mounted inside housing 10, in which case the housing can include a door or panel 11 which can be opened for access to a battery compartment for removal and replacement of the battery. In another embodiment the one or more batteries can be mounted externally to one or more sides of housing 10.

The system is controlled by a microprocessor based controller typically contained on a control board disposed within housing 10. A control panel 16 is provided on housing 10 and includes a display 18 such as a two digit alpha, numeric or alpha numeric digital display to indicate countdown of remaining time during a decontamination cycle and to indicate system messages such as error conditions. An audio annunciator 20 such as a Sonalert is provided to audibly indicate that an operating cycle has ended. The annunciator can also provide distinguishable sounds to denote one or more error conditions. A start or control switch 22 is provided to activate the system. The control switch can be of the illuminated type which illuminates when actuated to start a decontamination cycle. A USB port 32 is also coupled to the controller and enables the microprocessor to be programmed or reprogrammed via an external computer such as a PC.

The system can be operated remotely via a remote control 34 which communicates with the controller. The remote control typically contains the same controls and indicators as on the control panel 16. In an alternative embodiment the system may be operated only via a remote control, in which case no control panel would be provided on the housing. The remote communication between the remote control 34 and the controller can be wireless via radio, ultrasonic or optical signal as per se is known in the art. A wired remote control can alternatively be used.

The system in the illustrated embodiment including batteries weighs about ten pounds. In the illustrated embodiment the two U shaped lamps are mounted parallel to each other. An enclosure in the form of a protective metal or plastic grid or cage 13 is disposed over the pair of lamps for mechanical protection of the lamps. The grid is sufficiently open to not significantly shield radiation from the lamps. The protective grid can be retained in place on the housing by any convenient means such as a support post or fasteners at the bottom of the grid. Alternatively to an open grid structure the enclosure can be a solid cover of UVC transmissive material.

The housing is supported on a stand 30 such as a tripod stand. The stand may be collapsible for ease of transport and set up of the system in confined spaces such as an aircraft bathroom. The stand may also be adjustable in height to position the lamp for efficient radiation in the room.

A motion detector 24 is provided on one or more sidewalls of housing 10 to provide a signal to the system controller in the event that motion is detected such as would occur by a person moving into proximity with the system. If the system is activated when a person is present, the system, in response to a signal from one or more of the motion detectors, will shut down to prevent the person being exposed to UVC radiation. The motion sensors can be associated with one or more LEDs 21 on the control panel to usually indicate when motion has been detected. An audio alarm can also be provided by annunciator 20. Other embodiments can use a single motion sensor or multiple ones in selected positions on the housing.

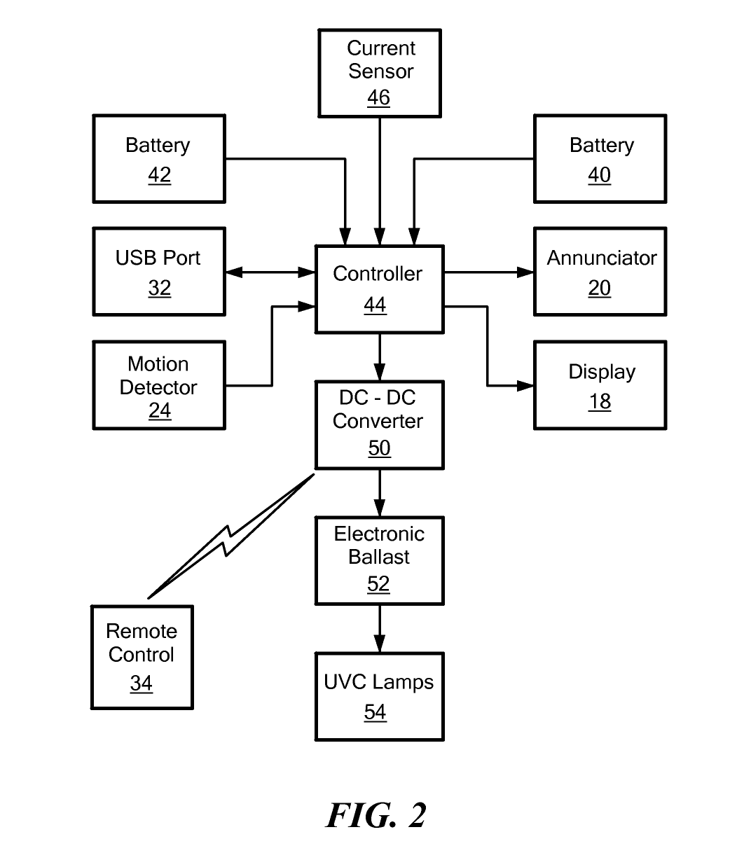

The system is shown in block diagram form in FIG. 2. Batteries 40 and 42 provide DC power to a control processor 44 which also receives a signal from a current sensor 46. Motion detector 24 provides signals to controller 44. The controller provides a control signal to DC to DC converter 50 which in turn drives lamp ballasts 52 which drive UVC lamps 54. The controller 44 also provides output signals to one or more system displays 18 and an audio annunciator 20. The system typically operates for a predetermined period of time as governed by a time period set in the controller. Upon activation of the system by pushing the start switch 22, the lamps are turned on for the specified period of time and are turned off when the time ends. A countdown of the operating time is shown in the display 18 on the front panel.

The controller 44 monitors the current to each of the electronic ballasts to insure that all of the lamps are operating properly. If the current as sensed by the current sensor 46 is less than the designated reference value, the controller will turn off the UVC lamps and display a message on front panel display 18. Typically, the current monitor signal is converted to a digital signal by means of an analog to digital converter for comparison with a stored reference value. The converter can be part of the controller or a separate item.

The batteries are typically lithium ion batteries rated at 28 volts and which can be recharged in approximately one hour. In the illustrated embodiment two UVC lamps are employed each having a 55 watt rating. The illustrated embodiment provides sufficient ultraviolet intensity to decontaminate a typical aircraft bathroom in three minutes and the battery supply is sufficient to decontaminate about ten bathrooms before the batteries need to be recharged.

The controller monitors the voltage of the batteries and provides a signal to the visual display 18 to indicate a low battery condition and serve as a reminder that it is time to recharge the batteries. Other visual and/or audible indications can be provided by LED 21 and annunciator 20 to warn of a low battery condition.

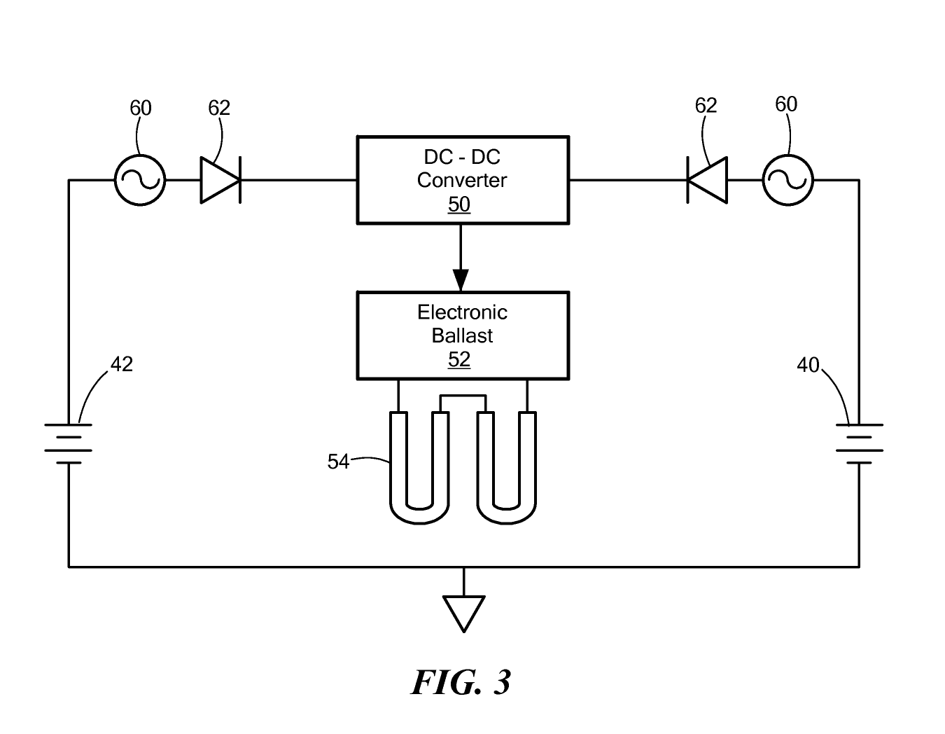

An electrical schematic of the system is shown in FIG. 3. Each battery has one terminal coupled to DC to DC converter 50 via an overcurrent protective device such as a thermally resettable circuit breaker 60, and an isolation diode 62. Each isolation diode is in series with the respective circuit breaker and prevents interaction between the batteries. The other battery terminals are coupled to electrical ground as shown. The batteries feed the converter 50 in parallel and the converter changes the battery voltage, typically 18-30 volts DC, to a fixed DC level of 120 volts. The electronic ballasts operate at 120 volts DC as well as 120 volts AC, and in the present embodiment the ballasts are driven by direct current from the batteries and converter. The current from the converter 50 into the ballasts is monitored typically in the ground lead to determine if one or more of the UVC lamps are operating properly. The monitored current is compared by the controller with a reference value and if below the reference value, the controller shuts down the system and causes indication of an error condition on display 18 and/or annunciator 20. In the illustrated embodiment, the UVC lamps are driven in series by the ballasts so that both tubes are either fully on or off. Alternatively, two separate ballasts could be used to drive each lamp individually.

The controller 44 governs the decontamination time of the system, monitors operating parameters of the system and provides visually and audio signals to indicate the operating conditions of the system. The microprocessor includes an analog to digital converter which monitors the current to the electronic ballasts, the voltage of each battery and the control signals from the start switch and motion detectors.

The embodiment shown is operative to kill at least 99% of pathogens including Clostridium difficile in about 3 minutes in an aircraft bathroom in which the surfaces to be decontaminated are no more than three feet from the UVC lamps.

The invention is not to be limited by the particular embodiments shown and modifications and alternative implementations are contemplated and within the intended scope of the invention. Accordingly, the invention is not to be limited by what has been particularly shown and described except as defined by the appended claims.

1. A portable battery powered ultraviolet radiation sanitation system for placement in a room to be sanitized, the system comprising:
a portable housing;
one or more UVC lamps providing UVC radiation and mounted in vertical orientation on the housing to emit UVC radiation to the room in which the system is placed;
a lamp enclosure for the one or more lamps providing mechanical protection of the lamps with no significant shielding of radiation from the one or more lamps into the room;
a power source disposed in the housing and having at least one electronic ballast to drive the one or more UVC lamps;
at least one battery coupled to the power source;
at least one DC-to-DC converter to convert a low battery voltage to a higher voltage sufficient to drive the at least one electronic ballast;
an electronic controller to control operation of the system including monitoring the current to the at least one electronic ballast, comparing the value with a predetermined value and turning off the system if the current is less than the predetermined value; and
at least one motion detector to turn off the UVC lamps in the event motion is detected in the vicinity of the lamps.

2. The system in claim 1 wherein the UVC lamps are approximately 20 inches in length and operate at 55 watts of power.

3. The system of claim 1 wherein the lamp enclosure is an open grid which does not significantly affect UVC radiation from the one or more UVC lamps.

4. The system of claim 1 including a remote control for communicating with the controller.

5. The system of claim 4 in which the remote control wirelessly communicates with the controller.

6. The system of claim 1 wherein the housing is supported by a portable stand.

7. (canceled)

8. (canceled)

9. The system of claim 1 wherein the controller has at least one analog-to-digital converter.

10. The system of claim 1 wherein the UVC lamps are low pressure high output mercury lamps.

11. The system of claim 1 wherein the UVC lamps are low pressure high output amalgam lamps.

12. The system of claim 1 wherein the lamps are U-shaped and have a connector at one end.

13. The system of claim 1 wherein each UVC lamp is enclosed in a protective sleeve of UVC transmissive material.

14. The system of claim 13 wherein the sleeve is FEP.

15. The system of claim 1 wherein the housing includes a control panel.

16. The system of in claim 15 where the control panel includes an alpha, numeric or alpha/numeric display, an audio annunciator and a push button start switch.

17. The system of claim 16 wherein the audio annunciator is a Sonalert.

18. The system of claim 16 wherein the push button start switch is lighted to indicate power is applied to the system.

19. The system of claim 16 wherein one push of the push button switch will start the on time of the UVC lamps and a second push will terminate operation of the UVC lamps and reset the control panel visual and/or audio indicators.

20. The system in claim 1 wherein the controller has a USB port to enable the microprocessor to be programmed from a computer.

21. The system in claim 1 wherein each battery has an over current protective device.

22. (canceled)

23. The system of claim 1 wherein there are two or more UVC lamps.

24. The system of claim 1 wherein there is sufficient UVC intensity to kill at least 99.9% of pathogens including Clostridium difficile in three minutes at a distance of 3 feet from the UVC lamps.

25. The system of claim 1 wherein the one or more batteries are Lithium-ion batteries.

26. The system of claim 23 in which two UVC lamps are connected in series and are driven by a single electronic ballast.

27. A portable battery powered ultraviolet radiation sanitation system comprising:
a housing;
a plurality of UVC lamps mounted on the housing and providing UVC radiation in a surrounding area;
a lamp enclosure for the plurality of lamps providing mechanical protection of the lamps and of an open grid construction to not significantly shield radiation from the lamps;
a pair of batteries;
a power source in the housing coupled to the batteries and having at least one electronic ballast to drive the UVC lamps;
a DC-to-DC converter coupled to each of the batteries via a respective overcurrent protective device and a series connected isolation diode, and operative to convert the low battery voltage to a higher voltage sufficient to drive the at least one electronic ballast; and
an electronic controller to control operation of the system including monitoring the current from the DC-to-DC converter to the at least one electronic ballast.

28. The system of claim 1 wherein the at least one battery includes a pair of batteries;
and wherein the at least one DC-to-DC converter is coupled to each of the batteries via a respective overcurrent protective device and a series connected isolation diode, and operative to convert the low battery voltage to a higher voltage sufficient to drive the at least one electronic ballast.
